(19) United States
(12) Patent Application Publication
(10) Pub. No.: US 2026/0325475 A1
(43) Pub. Date: Oct. 22, 2026

(54) FORMULATIONS FOR DRY EYES AND METHODS OF PREPARING AND USING THE SAME

(71) Applicant: Maravilla, LLC, Bloomfield Hills, MI (US)

(72) Inventors: Fernando PENICHE, Mexico City (MX); Maria Del Carmen MARTINEZ, Mexico City (MX); Jim STRINGHAM, Glendale, AZ (US); Jose TORRES QUIROGA, Monterrey (MX); Carlos TORRES GOMEZ, Monterrey (MX)

(73) Assignee: Maravilla, LLC, Bloomfield Hills, MI (US)

(21) Appl. No.: 19/468,450

(22) Filed: Feb. 3, 2026

Related U.S. Application Data
(60) Provisional application No. 63/753,174, filed on Feb. 3, 2025.

(51) Int. Cl. A61K 31/202; A61K 9/00; A61K 9/48; A61P 27/02

(52) U.S. Cl. CPC A61K 31/202; A61K 9/0053; A61K 9/4858; A61K 9/4875; A61P 27/02

(57) ABSTRACT

A formulation for supporting ocular health includes re-esterified eicosapentaenoic acid and re-esterified docosahexaenoic acid, where a weight ratio of the re-esterified eicosapentaenoic acid to the re-esterified docosahexaenoic acid is 1:1.

### CROSS-REFERENCE TO RELATED APPLICATIONS

This application claims the benefit of U.S. Provisional Application No. 63/753,174 filed on Feb. 3, 2025. The entire disclosure of the above application is incorporated herein by reference.

### FIELD

The present disclosure relates to formulations for dry eyes and methods of preparing and using the same.

### BACKGROUND

This section provides background information related to the present disclosure which is not necessarily prior art.

Tear dysfunction syndrome, also known as dry eye disease, is a multifactorial disease characterized by a loss of tear film homeostasis as accompanied by ocular symptoms include general discomfort, visual disturbance, and team film instability. Tear dysfunction syndrome often impacts quality of life by limiting various activities, including reading, outdoor recreation, driving, and computer work. Dietary supplementation with omega-3 polyunsaturated fatty acids, for example, as obtained from the oil of fatty fish (e.g., salmon, anchovy, sardine, mackerel), have been useful to address tear dysfunction syndrome and its symptoms.

There are three main omega-3 polyunsaturated fatty acids, including alpha-linolenic acid (ALA), eicosapentaenoic acid (EPA), and docosahexaenoic acid (DHA). Alpha-linolenic acid (ALA) is often found in oilseed plants such as flaxseed, soybeans, and canola oil. Eicosapentaenoic acid (EPA) and docosahexaenoic acid (DHA) are often found naturally in fish, especially cold-water fish such as salmon, mackerel, tuna, herring, and sardines, as well as in algae and shellfish. Eicosapentaenoic acid (EPA) and docosahexaenoic acid (DHA) as found naturally in fish is in a triglyceride form, where the fatty acids (i.e., eicosapentaenoic acid (EPA) and docosahexaenoic acid (DHA)) are stacked and bound to a natural alcohol backbone, glycerol. Industrial processing of fish oil often involves removing the fatty acids from glycerol and attaching them to a synthetic alcohol backbone, ethanol. This form of fish oil (i.e., as bound to ethanol) is referred to as ethyl ester.

The bioavailability of ethyl ester in the human body is less than the bioavailability of the natural form of the fish oil (i.e., as bound to glycerol) in the human body. The bioavailability of the ethyl ester may be improved by re-esterifying the ethyl ester by removing the eicosapentaenoic acid (EPA) and docosahexaenoic acid (DHA) from the synthetic backbone and attaching the same to a natural backbone, forming a re-esterified triglyceride.

### SUMMARY

This section provides a general summary of the disclosure, and is not a comprehensive disclosure of its full scope or all of its features.

In various aspects, the present disclosure provides a formulation for supporting ocular health.

In at least one example embodiment, the formulation may include eicosapentaenoic acid (EPA) and docosahexaenoic acid (DHA), where a weight ratio of the eicosapentaenoic acid (EPA) to the docosahexaenoic acid (DHA) is 1:1.

In at least one example embodiment, the eicosapentaenoic acid (EPA) may include re-esterified eicosapentaenoic acid (EPA) and the docosahexaenoic acid (DHA) may include re-esterified docosahexaenoic acid (DHA).

In at least one example embodiment, the formulation may include greater than or equal to about 15 weight percent to less than or equal to about 30 weight percent of the eicosapentaenoic acid (EPA) and greater than or equal to about 15 weight percent to less than or equal to about 30 weight percent of the docosahexaenoic acid (DHA).

In at least one example embodiment, the formulation may include greater than or equal to about 1,000 milligrams to less than or equal to about 1,500 milligrams of the eicosapentaenoic acid (EPA) and greater than or equal to about 1,000 milligrams to less than or equal to about 1,500 milligrams of the docosahexaenoic acid (DHA).

In at least one example embodiment, the formulation may further include greater than 0 weight % to less than or equal to about 50 weight % of a carrier.

In at least one example embodiment, the carrier may include monounsaturated fats and polyunsaturated fatty acids.

In at least one example embodiment, the carrier may further include vitamin A, vitamin D, or a combination of vitamin A and vitamin D.

In at least one example embodiment, the formulation may further include a flavorant.

In at least one example embodiment, the formulation may include greater than 0 weight percent to less than or equal to about 2 of the flavorant.

In at least one example embodiment, the flavorant may include lemon.

In at least one example embodiment, the formulation may further include an antioxidant.

In at least one example embodiment, the formulation may include greater than or equal to about 0 weight percent to less than or equal to about 2 of the antioxidant.

In at least one example embodiment, the antioxidant may include rosemary extract, sunflower oil, natural tocopherols, ascorbyl palmitate, or any combination thereof.

In at least one example embodiment, the formulation may further include two or more consumables, each consumable of the two or more consumables being the same and including the eicosapentaenoic acid (EPA) and the docosahexaenoic acid (DHA).

In various aspects, the present disclosure provides a method for supporting ocular health.

In at least one example embodiment, the method may include administering a formulation to a subject. The formulation may include eicosapentaenoic acid (EPA) and docosahexaenoic acid (DHA), where a weight ratio of the eicosapentaenoic acid (EPA) to the docosahexaenoic acid (DHA) is 1:1.

In at least one example embodiment, the eicosapentaenoic acid (EPA) may include re-esterified eicosapentaenoic acid (EPA) and the docosahexaenoic acid (DHA) may include re-esterified docosahexaenoic acid (DHA).

In at least one example embodiment, the formulation may include greater than or equal to about 15 weight percent to less than or equal to about 30 weight percent of the eicosapentaenoic acid (EPA) and greater than or equal to about 15 weight percent to less than or equal to about 30 weight percent of the docosahexaenoic acid (DHA).

In at least one example embodiment, the formulation may further include greater than 0 weight percent to less than or equal to about 2 of a flavorant.

In at least one example embodiment, the formulation may further include greater than or equal to about 0 weight percent to less than or equal to about 2 of an antioxidant.

In at least one example embodiment, the administering of the formulation to the subject may include administering two or more consumables to the subject, each consumable of the two or more consumables being the same and including the eicosapentaenoic acid (EPA) and the docosahexaenoic acid (DHA).

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a graphical demonstration summarizing ocular surface disease index scores over time for subjects ingesting formulations prepared in accordance with various aspects of the present disclosure.

FIG. 2 is another graphical demonstration summarizing ocular surface disease index scores over time for subjects ingesting formulations prepared in accordance with various aspects of the present disclosure.

FIG. 3 is a graphical demonstration summarizing tear breakup time over time for subjects ingesting formulations prepared in accordance with various aspects of the present disclosure.

FIG. 4 is a graphical demonstration summarizing fluorescein clearance test results for subjects ingesting formulations prepared in accordance with various aspects of the present disclosure.

FIG. 5 is a graphical demonstration summarizing further fluorescein clearance test results for subjects ingesting formulations prepared in accordance with various aspects of the present disclosure.

FIG. 6 is a graphical demonstration summarizing still further fluorescein clearance test results for subjects ingesting formulations prepared in accordance with various aspects of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Dietary formulations or compositions or supplements (commonly in the form of tablets, capsules, gels, liquids, and/or powders) are often consumed by human subjects to supplement and/or replace important vitamins and nutrients that are not otherwise sufficiently absorbed via consumed foods and/or to mitigate disease risk and symptoms thereof and/or to improve or support musculoskeletal function. In accordance with various aspects of the present disclosure, triglycerides (including, for example, eicosapentaenoic acid (EPA), docosahexaenoic acid (DHA), or a combination of eicosapentaenoic acid (EPA) and docosahexaenoic acid (DHA)), and more specifically, re-esterified triglycerides (including, for example, re-esterified eicosapentaenoic acid (EPA), re-esterified docosahexaenoic acid (DHA), or a combination of re-esterified eicosapentaenoic acid (EPA) and re-esterified docosahexaenoic acid (DHA)) are prescribed to and/or administered to and/or otherwise ingested or consumed by subjects (including human or non-human subjects) to improve ocular health (including, for example, Tear dysfunction syndrome). In addition to ocular health, triglycerides (including, for example, eicosapentaenoic acid (EPA), docosahexaenoic acid (DHA), or a combination of eicosapentaenoic acid (EPA) and docosahexaenoic acid (DHA)), and more specifically, re-esterified triglycerides (including, for example, re-esterified eicosapentaenoic acid (EPA), re-esterified docosahexaenoic acid (DHA), or a combination of re-esterified eicosapentaenoic acid (EPA) and re-esterified docosahexaenoic acid (DHA)) are prescribed to and/or administered to and/or otherwise ingested or consumed by subjects (including human or non-human subjects) to improve hair health, respiratory ease, joint mobility, skin texture and flexibility, nail growth and quality, medication absorption and responses, or any combination thereof.

In accordance with various aspects of the present disclosure, the re-esterified triglycerides may be prescribed/administered/ingested/consumed in the form of re-esterified triglyceride formulation. The formulation may include the re-esterified triglycerides in an amount ranging from greater than or equal to about 30 weight percent to less than or equal to about 60 weight percent (e.g., greater than or equal to about 32 weight percent to less than or equal to about 58 weight percent, greater than or equal to about 34 weight percent to less than or equal to about 56 weight percent, greater than or equal to about 36 weight percent to less than or equal to about 54 weight percent, greater than or equal to about 38 weight percent to less than or equal to about 52 weight percent, greater than or equal to about 40 weight percent to less than or equal to about 50 weight percent, greater than or equal to about 42 weight percent to less than or equal to about 48 weight percent, or greater than or equal to about 44 weight percent to less than or equal to about 46 weight percent).

In at least one example embodiment, the formulation includes re-esterified eicosapentaenoic acid (EPA), re-esterified docosahexaenoic acid (DHA), or a combination of re-esterified eicosapentaenoic acid (EPA) and re-esterified docosahexaenoic acid (DHA). The formulation may include a 1:1 weight ratio of eicosapentaenoic acid (EPA) and docosahexaenoic acid (DHA). For example, the formulation may include the omega-3 polyunsaturated fatty acids in an amount greater than or equal to about 2,000 milligrams to less than or equal to about 3,000 milligrams (e.g., greater than or equal to about 2,100 milligrams to less than or equal to about 2,900 milligrams, greater than or equal to about 2,200 milligrams to less than or equal to about 2,800 milligrams, greater than or equal to about 2,300 milligrams to less than or equal to about 2,700 milligrams, greater than or equal to about 2,400 milligrams to less than or equal to about 2,600 milligrams, or about 2,200 milligrams, or about 2560 milligrams), where the formulation includes of the re-esterified eicosapentaenoic acid (EPA) in an amount greater than or equal to about 1,000 milligrams to less than or equal to about 1,500 milligrams (e.g., greater than or equal to about 1,100 milligrams to less than or equal to about 1,400 milligrams, greater than or equal to about 1,200 milligrams to less than or equal to about 1,300 milligrams, about 1,100 milligrams, or about 1,280 milligrams) and the re-esterified docosahexaenoic acid (DHA) in an amount greater than or equal to about 1,000 milligrams to less than or equal to about 1,500 milligrams (e.g., greater than or equal to about 1,100 milligrams to less than or equal to about 1,400 milligrams, greater than or equal to about 1,200 milligrams to less than or equal to about 1,300 milligrams, about 1,100 milligrams, or about 1,280 milligrams).

In at least one example embodiment, the formulation may include a carrier. For example, the formulation may include other fatty acids, including monounsaturated fats and other polyunsaturated fatty acids, as well as minor components, like vitamins A and D. The re-esterified eicosapentaenoic acid (EPA) and re-esterified docosahexaenoic acid (DHA) may be embedded within the carrier. at least one example embodiment, the formulation may include the carrier in an amount greater than or equal to about 0 weight percent to less than or equal to about 50 weight percent (e.g., greater than or equal to about 2 weight percent to less than or equal to about 48 weight percent, greater than or equal to about 4 weight percent to less than or equal to about 46 weight percent, greater than or equal to about 6 weight percent to less than or equal to about 44 weight percent, greater than or equal to about 8 weight percent to less than or equal to about 42 weight percent, greater than or equal to about 10 weight percent to less than or equal to about 40 weight percent, greater than or equal to about 12 weight percent to less than or equal to about 38 weight percent, greater than or equal to about 14 weight percent to less than or equal to about 36 weight percent, greater than or equal to about 16 weight percent to less than or equal to about 34 weight percent, greater than or equal to about 18 weight percent to less than or equal to about 32 weight percent, greater than or equal to about 20 weight percent to less than or equal to about 30 weight percent, greater than or equal to about 22 weight percent to less than or equal to about 28 weight percent, greater than or equal to about 24 weight percent to less than or equal to about 26 weight percent). In at least one example embodiment, the formulation may include the carrier in an amount of about 267 milligrams.

In at least one example embodiment, the formulation may include a flavorant. For example, the formulation may include a lemon flavorant. In at least one example embodiment, the formulation may include the flavorant in an amount greater than or equal to about 0 weight percent to less than or equal to about 2 weight percent (e.g., greater than or equal to about 0.1 weight percent to less than or equal to about 2 weight percent, greater than or equal to about 0.2 weight percent to less than or equal to about 1.9 weight percent, greater than or equal to about 0.3 weight percent to less than or equal to about 1.8 weight percent, greater than or equal to about 0.4 weight percent to less than or equal to about 1.7 weight percent, greater than or equal to about 0.5 weight percent to less than or equal to about 1.6 weight percent, greater than or equal to about 0.6 weight percent to less than or equal to about 1.5 weight percent, greater than or equal to about 0.7 weight percent to less than or equal to about 1.4 weight percent, greater than or equal to about 0.8 weight percent to less than or equal to about 1.3 weight percent, greater than or equal to about 0.9 weight percent to less than or equal to about 1.2 weight percent, greater than or equal to about 1 weight percent to less than or equal to about 1.1 weight percent, or about 1 weight percent). In at least one example embodiment, the formulation may include the flavorant in an amount of about 16 milligrams. In at least one example embodiment, the formulation may include the flavorant in an amount of about 19 milligrams.

In at least one example embodiment, the formulation may include an antioxidant. For example, the formulation may include rosemary extract, sunflower oil, natural tocopherols, ascorbyl palmitate, or any combination thereof. In at least one example embodiment, the formulation may the antioxidant in an amount greater than or equal to about 0 weight percent to less than or equal to about 2 weight percent (e.g., greater than or equal to about 0.1 weight percent to less than or equal to about 2 weight percent, greater than or equal to about 0.2 weight percent to less than or equal to about 1.9 weight percent, greater than or equal to about 0.3 weight percent to less than or equal to about 1.8 weight percent, greater than or equal to about 0.4 weight percent to less than or equal to about 1.7 weight percent, greater than or equal to about 0.5 weight percent to less than or equal to about 1.6 weight percent, greater than or equal to about 0.6 weight percent to less than or equal to about 1.5 weight percent, greater than or equal to about 0.7 weight percent to less than or equal to about 1.4 weight percent, greater than or equal to about 0.8 weight percent to less than or equal to about 1.3 weight percent, greater than or equal to about 0.9 weight percent to less than or equal to about 1.2 weight percent, greater than or equal to about 1 weight percent to less than or equal to about 1.1 weight percent, or about 1 weight percent). In at least one example embodiment, the formulation may include the antioxidant in an amount of about 2.5 milligrams.

The formulation may be provided in the form of one or more consumables including, for example, tablets, capsules, gels, liquids, powders, or any combination thereof. For example, in various example embodiments, the formulation may be provided as two or more consumables, where each of the consumables include a portion of the formulation. In at least one example embodiment, each of the two or more consumables may have the same composition and structural configuration. In at least one example embodiment, the formulation may be provided as three consumables. In other example embodiments, the formulation may be provided as four consumables. However, it should be appreciated that the frequency of consumption and dosage of the formulation, and in turn, a number of consumables, may be adjusted to account for the concentration of the active agents and also the determined needs of the particular subject.

In at least one example embodiment, an example consumable includes a softgel capsule including a center (or core) and a shell defining the same, where the center may include the re-esterified triglycerides and a carrier and optionally an antioxidant, a flavorant, or a combination of the antioxidant and the flavorant. Like the formulation, the re-esterified triglycerides includes a 1:1 weight ratio of re-esterified eicosapentaenoic acid (EPA) and re-esterified docosahexaenoic acid (DHA). Further, like the formulation, the carrier may include other fatty acids, including monounsaturated fats and other polyunsaturated fatty acids, as well as minor components, like vitamins A and D. Further, like the formulation, the antioxidant may include, for example, rosemary extract, sunflower oil, natural tocopherols, ascorbyl palmitate, or any combination thereof. Like the formulation, the flavorant may include, for example, lemon. The shell may include gelatin, glycerin, and water, or any combination thereof in which the material(s) are provided in sufficient amounts to form the shell. In at least one example embodiment, the softgel capsules may further include a flavoring coating that surrounds (e.g., disposed on) at least a part exterior-facing surface of shell. The coating may be continuous or discontinuous. The flavorants defining the coating may be the same as or different from any flavorant(s) of the center.

In various aspects, the present disclosure provides methods to support ocular health. The method may include prescribing or administering to a subject a safe and therapeutically effective amount of the formulation. The formulation may be prescribed/administered/ingested/consumed daily. However, it should be appreciated that the frequency of consumption and dosage may be adjusted to account for the concentration of the active agents and also the determined needs of the particular subject. As used herein, the term “therapeutically effective amount” means an amount of a compound that, when administered to a subject is sufficient, either alone or in combination with additional therapies, to effectively support ocular health. The “therapeutically effective amount” may vary depending on, for example, the compound, pharmaceutical composition or pharmaceutical dosage form, the condition treated and its severity, and the age and weight of the patient to be treated.

Certain features of the current technology are further illustrated in the following non-limiting examples.

### Examples

Example formulations can be prepared in accordance with various aspects of the present disclosure. For example, an example formulation may include greater than or equal to about 1,650 milligrams to less than or equal to about 2,200 milligrams of omega-3 polyunsaturated fatty acids, which includes greater than or equal to about 825 milligrams to less than or equal to about 1,100 milligrams of re-esterified eicosapentaenoic acid (EPA) and greater than or equal to about 825 milligrams to less than or equal to about 1,100 milligrams of re-esterified docosahexaenoic acid (DHA) and a weight ratio of re-esterified eicosapentaenoic acid (EPA) and re-esterified docosahexaenoic acid (DHA) is 1:1.

The example formulation may also include a carrier wherein the omega-3 polyunsaturated fatty acids are embedded. The example formulation may include greater than or equal to about 801 milligrams to less than or equal to about 1,068 milligrams. The carrier may include other fatty acids, including monounsaturated fats and other polyunsaturated fatty acids, as well as minor components such as vitamins A and D. The example formulation may also include greater than or equal to about to about 7.5 milligrams to less than or equal to about 10 milligrams of an antioxidant. The antioxidant may include, for example, rosemary extract, sunflower oil, natural tocopherols, ascorbyl palmitate, or any combination thereof. The example formulation may also include greater than or equal to about 48 milligrams to less than or equal to about 76 milligrams of a flavorant. The flavorant may include lemon. The example formulation may also include gelatin, glycerin, and water, or any combination thereof.

Table 1 summarizes compositional details of an example consumable, for example, an example softgel consumable, where the formulation is defined by the combination of three to four consumables and where q.s. refers to an amount that is as much as is sufficient and ** is an approximation.

TABLE 1
Composition of Example Softgel Consumable

| | | | Milli- | |
|---|---|---|---|---|
| Structure | Material | | grams | |
| Center | Omega-3 | Eicosapentaenoic Acid | 275 | 612.5 |
| | Polyun- | (EPA) | | |
| | saturated | Docosahexaenoic Acid | 275 | |
| | Fatty | (DHA) | | |
| | Acids | Other (e.g., Alpha- | 62.5 | |
| | Carrier | Linolenic Acid) | | |
| | | Other Fatty Acids (e.g., | 267 | |
| | | monounsaturated fats, | | |
| | | other polyunsaturated | | |
| | | fatty acids) | | |
| | | Minor Components (e.g., | | |
| | | vitamins A and D) | | |
| | Anti- | Natural blend including | 2.5 | |
| | oxidant | rosemarye xtract, | | |
| | | sunflower oil, natural | | |
| | | tocopherols, | | |
| | | ascorbyl palmitate | | |

| | | |
|---|---|---|
| Flavorant | Lemon | 16 |

| Target Fill Weight | | 898 |
|---|---|---|
| Shell | Gelatin** | q.s. |
| | Glycerin** | q.s. |
| | Purified Water** | q.s. |
| Target Shell Weight*** | | 395 |
| Target Gross Weight of Consumable** | | 1,293 |
| Coating** | Lemon | 3 |

Subjects between ages 18 and 85 years with mild, moderate, or severe dry eye as diagnosed according to the ocular surface disease index (“OSDI”) questionnaire ingested the formulation as assigned for three months. Male subjects ingested four soft-gel tablets defined by the formulation per day over the three-month period. Female subject ingested three soft-gel tablets defined by the formulation per day over the three-month period. The formulation as defining each soft-gel tablet was the same. Ocular surface disease index, tear breakup times (“TRL”), and fluorescein clearance tests (“FAP”) were evaluated throughout the test period. Results were consistently compared to previous results regardless of the nature of the previous results (i.e., high or low).

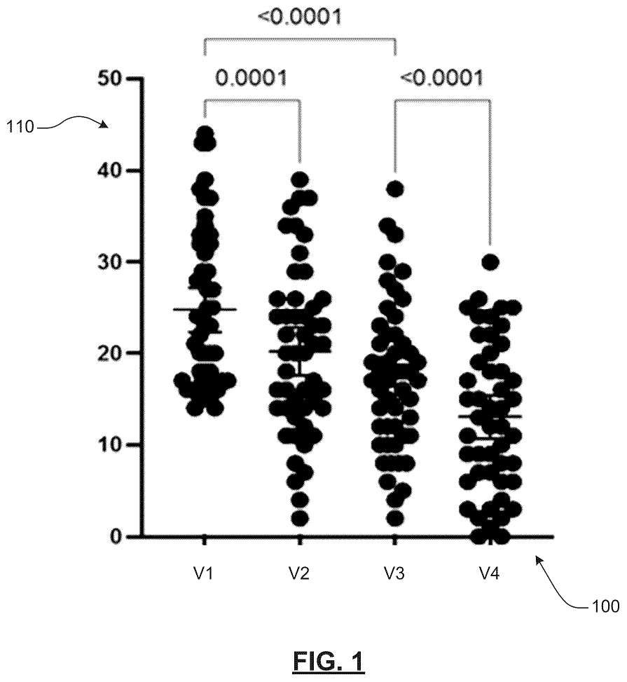

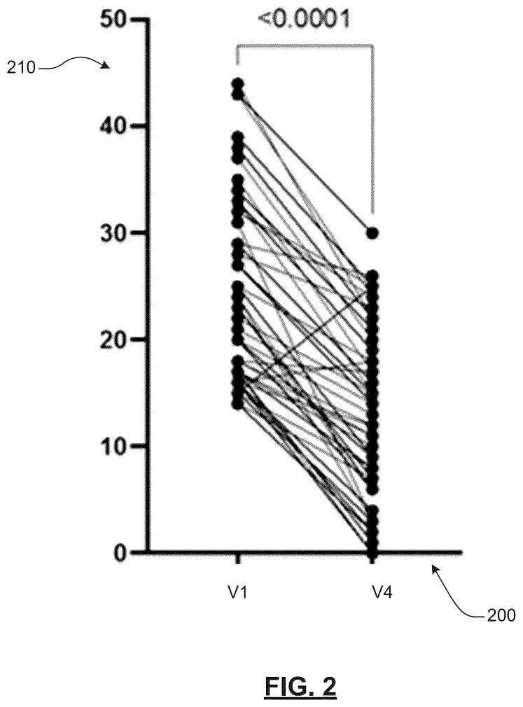

FIG. 1 is a graphical demonstration summarizing the ocular surface disease index scores over time for subjects ingesting the formulation, where the x-axis 100 represents time and the y-axis 102 represents ocular surface disease index scores, and where V1 represents ocular surface disease index scores at intake, V2 represents ocular surface disease index scores at 30 days, V3 represents ocular surface disease index scores at 60 days, and V4 represents ocular surface disease index scores at 90 days. FIG. 2 is a graphical demonstration further illustrating the ocular surface disease index scores between V1 and V2, where the x-axis 200 represents time and the y-axis 202 represents ocular surface disease index scores. As illustrated, and also further summarized in Table 2 below, the average inflammatory score during the pre-treatment assessment (V1) was 24.76, the average inflammatory score at 30 days (V2) was 20.02, the average inflammatory score at 60 days (V3) was 17.26, and the average inflammatory score at 90 days was 13.06. Statistically significant differences were found between the ocular surface disease index scores at V1 and V2, V3, and V4 and 91.3 percent of subjects showed some degree of improvement in Ocular Surface Disease Index Scores. Between intake (V1) and 30 days (V2) there was no statistically significant difference; however, between 30 days (V2) and 90 days (V4) the percentage of group improvement was 52.47 percent, an exponential report with statistical significance. The improvement was consistent between men and women, with no statistically significant differences found between the right and left eyes.

TABLE 2
Ocular Surface Disease Index Scores

| | Intake | 30 Days | 60 Days | 90 Days |
|---|---|---|---|---|
| Number of | 50 | 50 | 50 | 48 |
| Values | | | | |
| Minimum | 14 | 2 | 2 | 0 |
| 25 percent | 17 | 14 | 11 | 6.25 |
| Percentile | | | | |
| Median | 23 | 20 | 17 | 12.5 |
| 75 percent | 32 | 26 | 22 | 19.75 |
| Percentile | | | | |
| Maximum | 44 | 39 | 38 | 30 |
| Range | 30 | 37 | 36 | 30 |
| Mean | 24.76 | 20.2 | 17.26 | 13.06 |
| Standard | 8.7 | 9.123 | 8.061 | 8.107 |
| Deviation | | | | |
| Standard Error | 1.23 | 1.29 | 1.14 | 1.17 |
| of Mean | | | | |
| Coefficient of | 35.15 | 45.16 | 46.70 | 62.06 |
| Variation | percent | percent | percent | percent |
| Skewness | 0.6436 | 0.2202 | 0.4377 | 0.1615 |
| Kurtosis | −0.6937 | −0.5402 | −0.05491 | −1.013 |

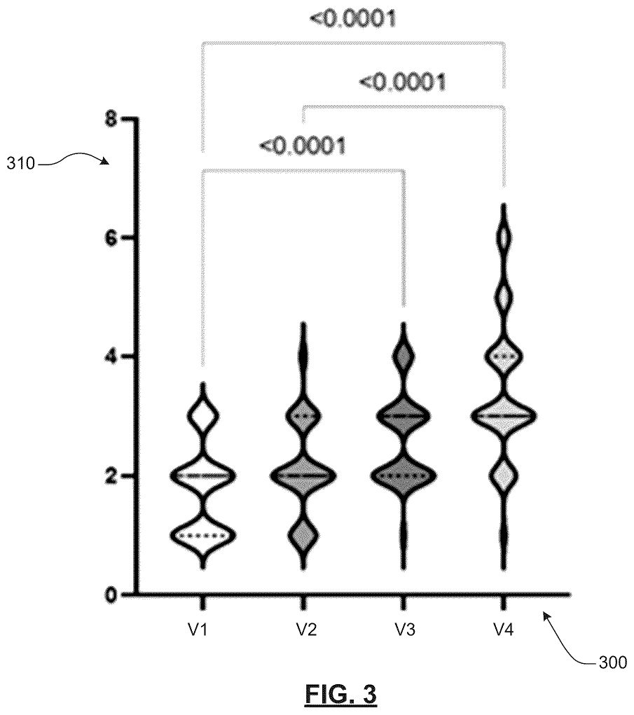

FIG. 3 is a graphical demonstration summarizing the tear breakup time over time for subjects ingesting the formulation, where the x-axis 300 represents time and the y-axis 302 represents tear breakup time, and where again V1 represents ocular surface disease index scores at intake, V2 represents ocular surface disease index scores at 30 days, V3 represents ocular surface disease index scores at 60 days, and V4 represents ocular surface disease index scores at 90 days. As illustrated, and also further summarized in Table 3 below, the average tear breakup time during the pre-treatment assessment (V1) was 1.77 seconds, the average tear breakup time at 30 days (V2) was 2.12 seconds, the average tear breakup time at 60 days (V3) was 2.62 seconds, and the average tear breakup time at 90 days (V4) was 3.42. A statistically significant increase was found in the tear breakup times at 60 days (V3). Between intake (V1) and 30 days (V2) there was a change of 0.35 seconds or 19.7 percent. Between intake (V1) and 90 days (V4) there was a change of 3.42 seconds or 193 percent. The improvement was consistent between men and women, with no statistically significant differences found between the right and left eyes.

TABLE 3
Tear Breakup Times

| | Intake | 30 Days | 60 Days | 90 Days |
|---|---|---|---|---|
| Number of | 100 | 100 | 98 | 96 |
| Values | | | | |
| Minimum | 1 | 1 | 1 | 1 |
| 25 percent | 1 | 2 | 2 | 3 |
| Percentile | | | | |
| Median | 2 | 2 | 3 | 3 |
| 75 percent | 2 | 3 | 3 | 4 |
| Percentile | | | | |
| Maximum | 3 | 4 | 4 | 6 |
| Range | 2 | 3 | 3 | 5 |
| Mean | 1.77 | 2.12 | 2.622 | 3.427 |
| Standard | 0.7366 | 0.7821 | 0.7394 | 1.158 |
| Deviation | | | | |
| Standard Error | 0.07366 | 0.07821 | 0.07469 | 0.1182 |
| of Mean | | | | |
| Coefficient of | 41.61 | 36.89 | 28.19 | 33.80 |
| Variation | percent | percent | percent | percent |
| Skewness | 0.394 | 0.3022 | 0.4254 | 0.5544 |
| Kurtosis | −1.061 | −0.282 | −0.5879 | 0.02405 |

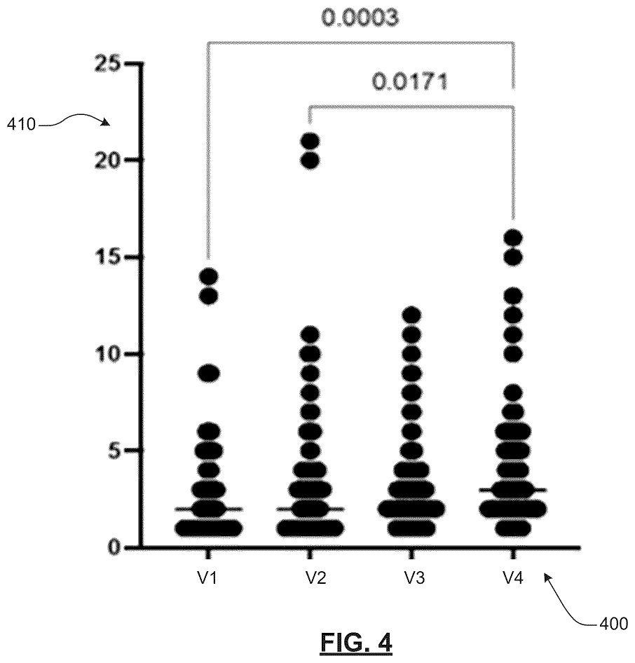

FIG. 4 is a graphical demonstration summarizing the fluorescein clearance tests over time for subjects ingesting the formulation, where the x-axis 400 represents time and the y-axis 402 represents average tear secretion (mm), and where V1 represents average tear secretion at 10 minutes pre-treatment, V2 represents average tear secretion at 30 days, V3 represents average tear secretion at 60 days, and V4 represents average tear secretion at 90 days. As illustrated, and also further summarized in Table 4 below, the average tear secretion during the pre-treatment assessment (V1) was 2.76 millimeters (mm), the average tear secretion at 30 days (V2) was 3.25 millimeters, the average tear secretion at 60 days (V3) was 3.08 millimeters, and the average tear secretion at 90 days (V4) was 3.91 millimeters. A statistically significant change was observed between the average tear secretion at V1 and V3 and V4. Between intake (V1) and 30 days (V2) there was an average increase of 0.46 millimeters or 16 percent in basal tear secretion. Between intake (V1) and 60 days (V3) there was an average increase of 1.12 millimeters or 40.14 percent in basal tear secretion. The improvement was consistent between men and women, with no statistically significant differences found between the right and left eyes.

Table 4
Fluorescein Clearance Tests, 10 minutes

| | −10 Minutes | 30 Days | 60 Days | 90 Days |
|---|---|---|---|---|
| Number of | 100 | 100 | 98 | 95 |
| Values | | | | |
| Minimum | 1 | 1 | 1 | 1 |
| 25 percent | 1 | 1 | 2 | 2 |
| Percentile | | | | |
| Median | 2 | 2 | 2 | 3 |
| 75 percent | 3.75 | 4 | 4 | 5 |
| Percentile | | | | |
| Maximum | 14 | 21 | 12 | 16 |
| Range | 13 | 20 | 11 | 15 |
| Mean | 2.79 | 3.25 | 3.082 | 3.916 |
| Standard | 2.552 | 3.392 | 2.282 | 2.956 |
| Deviation | | | | |
| Standard Error | 0.2552 | 0.3392 | 0.2306 | 0.3033 |
| of Mean | | | | |
| Coefficient of | 91.46 | 104.4 | 74.07 | 75.49 |
| Variation | percent | percent | percent | percent |
| Skewness | 2.138 | 3.105 | 1.926 | 2.005 |
| Kurtosis | 5.477 | 12.41 | 3.884 | 4.827 |

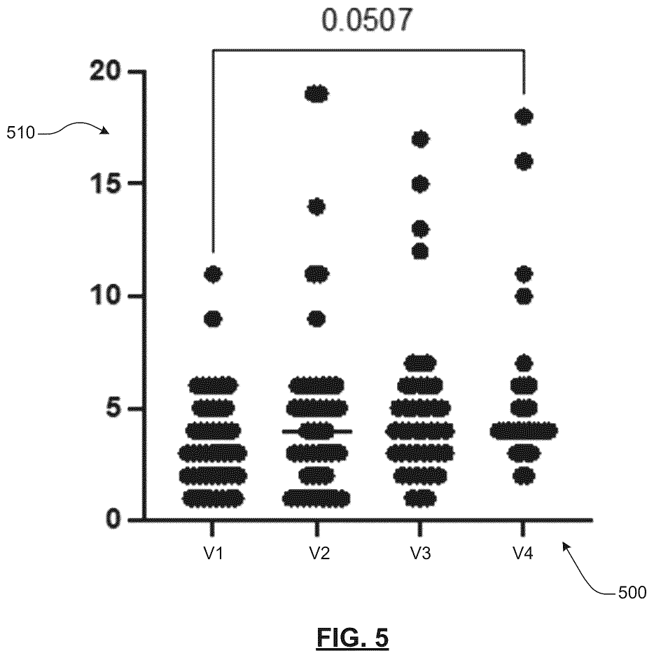

FIG. 5 is a graphical demonstration summarizing the fluorescein clearance tests over time for subjects ingesting the formulation, where the x-axis 500 represents time and the y-axis 502 represents average tear secretion, and where V1 represents average tear secretion at 20 minutes pre-treatment, V2 represents average tear secretion at 30 days, V3 represents average tear secretion at 60 days, and V4 represents average tear secretion at 90 days. As illustrated, and also further summarized in Table 5 below, the average tear secretion during the pre-treatment assessment (V1) was 3.52 millimeters, the average tear secretion at 30 days (V2) was 4.82 millimeters, the average tear secretion at 60 days (V3) was 4.86 millimeters, and the average tear secretion at 90 days (V4) was 5.53 millimeters. No statistically significant change was observed.

Table 5
Fluorescein Clearance Tests, 20 minutes

| | −20 Minutes | 30 Days | 60 Days | 90 Days |
|---|---|---|---|---|
| Number of | 46 | 46 | 44 | 28 |
| Values | | | | |
| Minimum | 1 | 1 | 1 | 2 |
| 25 percent | 2 | 2 | 3 | 4 |
| Percentile | | | | |
| Median | 3 | 4 | 4 | 4 |
| 75 percent | 5 | 6 | 6 | 6 |
| Percentile | | | | |
| Maximum | 11 | 19 | 17 | 18 |
| Range | 10 | 18 | 16 | 16 |
| Mean | 3.522 | 4.826 | 4.864 | 5.536 |
| Standard | 2.158 | 4.17 | 3.461 | 3.825 |
| Deviation | | | | |
| Standard Error | 0.3181 | 0.6149 | 0.5218 | 0.7228 |
| of Mean | | | | |
| Coefficient of | 61.26 | 86.41 | 71.17 | 69.09 |
| Variation | percent | percent | percent | percent |
| Skewness | 1.225 | 2.043 | 1.996 | 2.241 |
| Kurtosis | 2.24 | 4.622 | 4.318 | 4.843 |

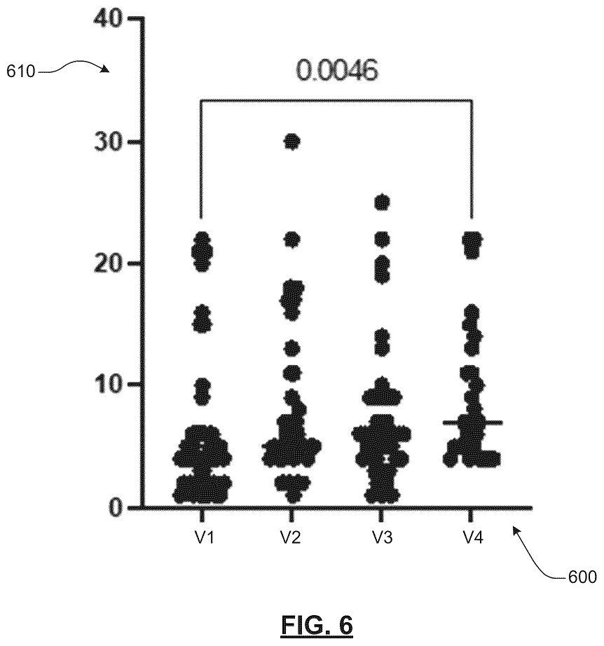

FIG. 6 is a graphical demonstration summarizing the fluorescein clearance tests over time for subjects ingesting the formulation, where the x-axis 600 represents time and the y-axis 602 represents average tear secretion (mm), and where V1 represents average tear secretion at 30 minutes pre-treatment, V2 represents average tear secretion at 30 days, V3 represents average tear secretion at 60 days, and V4 represents average tear secretion at 90 days. As illustrated, and also further summarized in Table 6 below, the average tear secretion during the during the pre-treatment assessment (V1) was 6.06 millimeters, the average tear secretion at 30 days (V2) was 7.50 millimeters, the average tear secretion at 60 days (V3) was 7.20 millimeters, and the average tear secretion at 90 days (V4) was 9.10 millimeters. A statistically significant change was observed between the average tear secretion at V1 and V4. The improvement was consistent between men and women, with no statistically significant differences found between the right and left eyes.

Table 6
Fluorescein Clearance Tests, 30 minutes

| | −30 Minutes | 30 Days | 60 Days | 90 Days |
|---|---|---|---|---|
| Number of | 46 | 46 | 44 | 28 |
| Values | | | | |
| Minimum | 1 | 1 | 1 | 4 |
| 25 percent | 2 | 4 | 4 | 5 |
| Percentile | | | | |
| Median | 4 | 5 | 6 | 7 |
| 75 percent | 6 | 8.25 | 9 | 12.5 |
| Percentile | | | | |
| Maximum | 22 | 30 | 25 | 22 |
| Range | 21 | 29 | 24 | 18 |
| Mean | 6.065 | 7.5 | 7.205 | 9.107 |
| Standard | 5.829 | 6.043 | 5.437 | 5.679 |
| Deviation | | | | |
| Standard Error | 0.8594 | 0.891 | 0.8197 | 1.073 |
| of Mean | | | | |
| Coefficient of | 96.10 | 80.58 | 75.47 | 62.35 |
| Variation | percent | percent | percent | percent |
| Skewness | 1.694 | 1.842 | 1.767 | 1.197 |
| Kurtosis | 1.871 | 3.584 | 3.069 | 0.4226 |

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A formulation for supporting ocular health, the formulation comprising:
eicosapentaenoic acid (EPA); and
docosahexaenoic acid (DHA),
a weight ratio of the eicosapentaenoic acid (EPA) to the docosahexaenoic acid (DHA) being 1:1.

2. The formulation of claim 1, wherein the eicosapentaenoic acid (EPA) includes re-esterified eicosapentaenoic acid (EPA) and the docosahexaenoic acid (DHA) includes re-esterified docosahexaenoic acid (DHA).

3. The formulation of claim 1, wherein the formulation includes
greater than or equal to about 15 weight percent to less than or equal to about 30 weight percent of the eicosapentaenoic acid (EPA), and
greater than or equal to about 15 weight percent to less than or equal to about 30 weight percent of the docosahexaenoic acid (DHA).

4. The formulation of claim 1, wherein the formulation includes
greater than or equal to about 1,000 milligrams to less than or equal to about 1,500 milligrams of the eicosapentaenoic acid (EPA), and
greater than or equal to about 1,000 milligrams to less than or equal to about 1,500 milligrams of the docosahexaenoic acid (DHA).

5. The formulation of claim 1, further comprising:
greater than 0 weight % to less than or equal to about 50 weight % of a carrier.

6. The formulation of claim 5, wherein the carrier includes monounsaturated fats and polyunsaturated fatty acids.

7. The formulation of claim 6, wherein the carrier further includes vitamin A, vitamin D, or a combination of vitamin A and vitamin D.

8. The formulation of claim 1, further comprising:
a flavorant.

9. The formulation of claim 8, wherein the formulation includes greater than 0 weight percent to less than or equal to about 2 of the flavorant.

10. The formulation of claim 8, wherein the flavorant includes lemon.

11. The formulation of claim 1, further comprising:
an antioxidant.

12. The formulation of claim 11, wherein the formulation includes greater than or equal to about 0 weight percent to less than or equal to about 2 of the antioxidant.

13. The formulation of claim 11, wherein the antioxidant includes rosemary extract, sunflower oil, natural tocopherols, ascorbyl palmitate, or any combination thereof.

14. The formulation of claim 1, further comprising:
two or more consumables, each consumable of the two or more consumables being the same and including the eicosapentaenoic acid (EPA) and the docosahexaenoic acid (DHA).

15. A method for supporting ocular health, the method comprising:
administering a formulation to a subject, the formulation including
eicosapentaenoic acid (EPA); and
docosahexaenoic acid (DHA),
a weight ratio of the eicosapentaenoic acid (EPA) to the docosahexaenoic acid (DHA) being 1:1.

16. The method of claim 15, wherein the eicosapentaenoic acid (EPA) includes re-esterified eicosapentaenoic acid (EPA) and the docosahexaenoic acid (DHA) includes re-esterified docosahexaenoic acid (DHA).

17. The method of claim 15, wherein the formulation includes
greater than or equal to about 15 weight percent to less than or equal to about 30 weight percent of the eicosapentaenoic acid (EPA), and
greater than or equal to about 15 weight percent to less than or equal to about 30 weight percent of the docosahexaenoic acid (DHA).

18. The method of claim 15, wherein the formulation further includes greater than 0 weight percent to less than or equal to about 2 of a flavorant.

19. The method of claim 15, wherein the formulation further includes greater than or equal to about 0 weight percent to less than or equal to about 2 of an antioxidant.

20. The method of claim 15, wherein the administering of the formulation to the subject includes administering two or more consumables to the subject, each consumable of the two or more consumables being the same and including the eicosapentaenoic acid (EPA) and the docosahexaenoic acid (DHA).
